(19) United States
(12) Patent Application Publication
(10) Pub. No.: US 2005/0107665 A1
(43) Pub. Date: May 19, 2005

(54) Device for sealing a body canal and method of use

(76) Inventor: Nady E. Nady, (US)

Correspondence Address: Gregory C. Houghton, Esq.;Mathews, Collins, Shepherd & McKay, P.A., Suite 306, 100 Thanet Circle, Princeton, NJ, 08540, US

(21) Appl. No.: 10/715,657

(22) Filed: Nov. 18, 2003

(51) Int. Cl. A61B 1/00

(52) U.S. Cl. 600/116; 606/193

(57) ABSTRACT

A device useful in diagnostic or surgical procedures for sealing the cervical canal in which an inflatable device is used with a hysteroscope instrument. The inflatable device provides a seal between an outer surface of the hysteroscope instrument and the cervical canal, thereby preventing fluids from exiting the cervical canal.

### BACKGROUND OF THE INVENTION

1. Field of the Invention

The present invention relates to a device useful in diagnostic or surgical procedures for sealing a cervical canal in which an inflatable device is used with a hysteroscope instrument. In an inflated position, the inflatable device provides a seal between an outer surface of the hysteroscope instrument and the cervical canal, thereby preventing fluids from exiting the cervical canal.

2. Related Art

Procedures which require entry into the uterus are well known. Two such procedures are diagnostic and surgical hysteroscopy.

Diagnostic hysteroscopy is a procedure used to help diagnose abnormal uterine bleeding, infertility, uterine and cervical cancer, location of intra uterine devices, complicated abortion and fetal examination. Diagnostic hysteroscopy involves inserting a hysteroscope instrument, which is a thin telescope, through the cervix into the uterus where the endometrial cavity (inside the uterus) can be observed.

Surgical hysteroscopy is a procedure used to remove polyps, cut adhesions, and other surgical procedures. Surgical hysteroscopy employs a type of hysteroscope instrument having channels through in which it is possible to insert very thin instruments to allow surgical procedures to occur within the uterus.

Because the inside of the uterus is a potential cavity, like a collapsed air dome, it is necessary to fill (distend) the uterus with either a fluid or a gas in order to visualize the interior of the uterus during both diagnostic and surgical hysteroscopy. However, the fluid or gas received in the uterus during either diagnostic or surgical hysteroscopy can subsequently flow from the uterus through the cervical canal resulting in a loss of pressure and an incapability of determining the amount of fluid or gas used in the procedure.

It is therefore desirable to provide a device to effectively seal the area between the outer surface of the hysteroscope instrument and the cervical canal, thereby preventing backflow of fluid or gas used to pressurize the uterus during either diagnostic or surgical hysteroscopy.

### SUMMARY OF THE INVENTION

The present invention provides a device for sealing an opening of a cervical canal during use of a hysteroscope instrument comprising an inflatable housing adapted to be received on an outer portion of a tubular body of the hysteroscope instrument. The inflatable housing surrounds an outer diameter of the outer portion of the tubular body of the hysteroscope instrument. The device includes means for inflating the inflatable housing, wherein when inflated the inflatable housing provides a seal between the outer portion of the tubular body of the hysteroscope instrument and a wall of the cervical canal.

Another embodiment of the present invention relates to a method for using the device for sealing a cervical canal during use of a hysteroscope instrument. In the method, fluid or gas is prevented from flowing out of a cervical canal from the uterus during the use of a hysteroscope instrument, using the steps of:
placing a device with means for inflating the device over the tubular portion of the hysteroscope instrument;
inserting the hysteroscope instrument into the cervical canal of the uterus so that the device is positioned within the cervical canal and an outer portion of the device is adjacent to the walls of the cervical canal;
connecting the means for inflating the device to an instrument that will inflate the device;
inflating the device thereby forming a seal between the outer portion of the device and the cervical canal; and
preventing fluid or gas from the uterus from flowing out of the cervical canal during the use of the hysteroscope instrument.

### BRIEF DESCRIPTION OF THE DRAWINGS

The advantage, nature, and various additional features of the invention will appear more fully upon consideration of the illustrative embodiments now to be described in detail in connection with accompanying drawings wherein:

FIG. 1 is a schematic diagram of a device for sealing a body canal according to an exemplary embodiment of the invention.

FIG. 2 is a schematic diagram of the device of the present invention on a hysteroscope instrument inserted into a cervical canal of a subject uterus.

FIG. 3 is a schematic diagram of an alternate embodiment of a device for sealing a body canal in which the device is permanently connected or integral with a hysteroscope sleeve.

It should be understood that the drawings are for purposes of illustrating the concept of the invention and are not necessarily to scale.

### DETAILED DESCRIPTION OF THE INVENTION

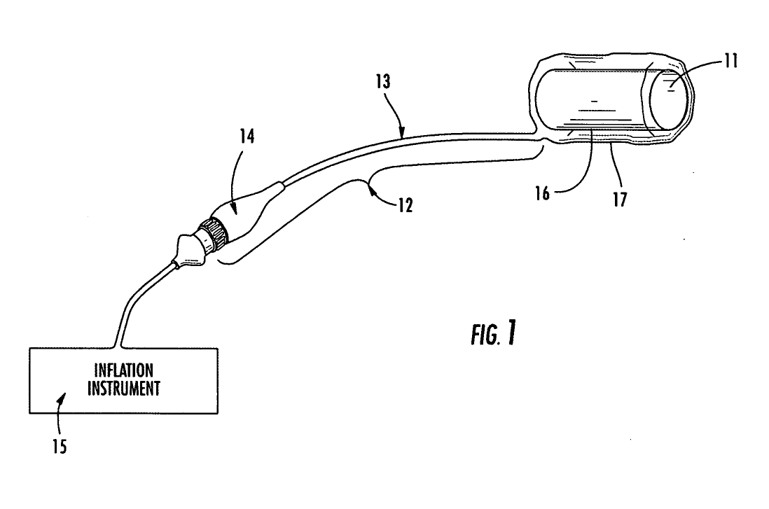

Referring to the drawings wherein like reference numerals identify similar or like elements throughout the several views and initially to FIG. 1, there is shown device 10 according to an exemplary embodiment of the invention. Device 10 includes an inflatable housing 11. Inflatable housing 11 has an open ended tubular shape. Inflatable housing 11 can be received around a tubular portion of a diagnostic or surgical instrument, as described below. Device 10 includes means for inflation 12 which is located outside a body canal during operation of device 10. Means for inflation 12 can include appendage 13 which attaches and/or locks using connector 14 to inflation instrument 15 to enable device 10 to be inflated. The instrument for inflation provides either air, inert gas or a solution through appendage 13. For example, inflation instrument 15 can be a syringe, pumping device or a one way pumping bulb. Inflatable housing 11 can be formed of a plurality of layers. For example, inflatable housing 11 can be formed of a rigid inner layer 16 and outer layer 17.

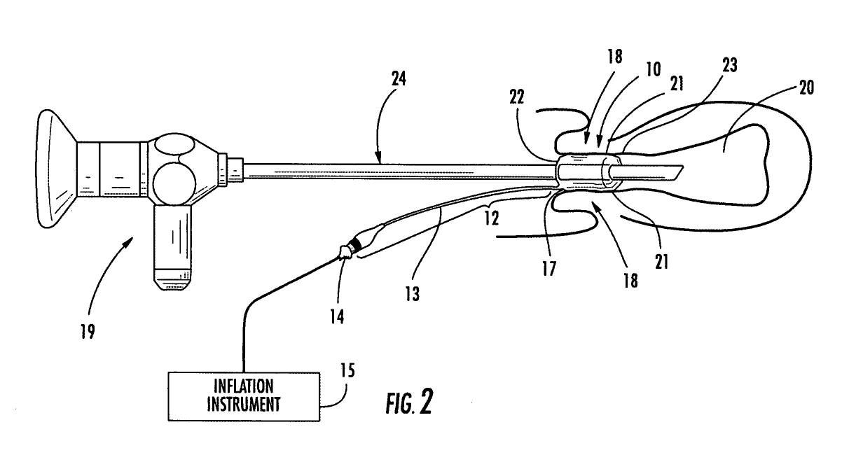

FIG. 2 schematically illustrates device 10 adapted to receive a hysteroscope instrument 19. Device 10 is positioned on tubular portion 24 of hysteroscope instrument 19 and is freely movable on tubular portion 24 of hysteroscope instrument 19. Thereafter, hysteroscope instrument 19 is positioned within cervical canal 18. Means for inflation 12 is positioned outside of cervical canal 18 and is connected to inflation instrument 15 to enable inflation of device 10. Device 10 upon being inflated forms seal 21 between outer portion 17 of device 10 and walls 23 of cervical canal 18 and forms seal 22 between inner part 16 of device 10 and tubular portion 24 of hysteroscope instrument 19. Seal 21 and seal 22 prevents backflow of gas or fluid from uterine cavity 20 through cervical canal 18.

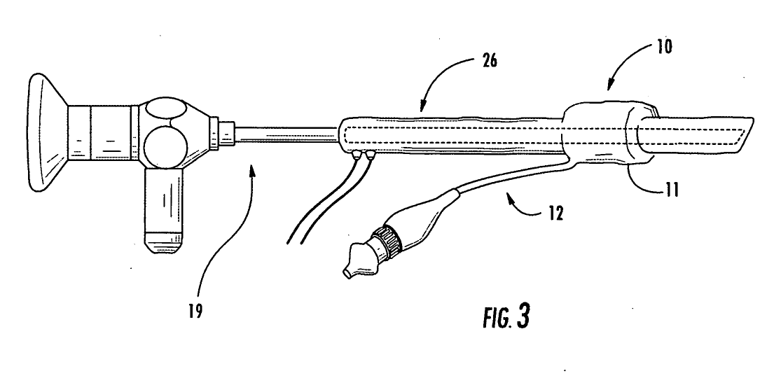

In an alternate embodiment shown in FIG. 3, device 10 is permanently attached or integral with hysteroscope sleeve 26. A hysteroscope sleeve as understood by those skilled in the art is an outer sheath designed particularly for a hysteroscope instrument that makes it possible to create a continuous flow system which can be used for controlled irrigation of the uterine cavity.

Device 10 can be formed from an elastomeric material such as polyurethane, poly(vinyl chloride) or any other suitable material. Inner layer 16 of device 10 is more rigid to allow for the integrity of device 10 when it is slid onto hysteroscope instrument 19.

The term fluid or gas include but are not limited to water, saline, isotonic solution, glycine solutions, carbon dioxide, or any other fluid or gas that would be appreciated by those skilled in the art as to be employed in hysteroscopy.

Although device 10 has been described for use associated with entry into the uterine cavity, one of ordinary skill in the art will recognize its usefulness in other related procedures.

Further, while the foregoing invention has been described with reference to the above embodiments, various modifications and changes can be made without departing from the spirit of the invention. Accordingly, all such modifications and changes are considered to be within the scope of the appended claims.

1. A device for sealing an opening of a body canal during use of a diagnostic or surgical instrument comprising:
an inflatable housing adapted to be received on an outer portion of a tubular body of said diagnostic or surgical instrument, said inflatable housing surrounding an outer diameter of said outer portion of said tubular body of said diagnostic or surgical instrument; and
means for inflating said inflatable housing, wherein when inflated said inflatable housing provides a seal between the outer portion of said tubular body of said diagnostic or surgical instrument and a wall of said body canal.

2. The device according to claim 1 wherein said means for inflating said inflatable housing is locating outside said body canal.

3. The device according claim 1 wherein said device is made from an elastomeric material.

4. The device according to claim 3 wherein said device comprises a plurality of layers.

5. The device according to claim 4 wherein said plurality of layers comprises a rigid inner layer and an outer layer, said rigid inner layer contacts the outer portion of said tubular body of said diagnostic or surgical instrument and said outer layer contacts said body canal.

6. The device according to claim 5 wherein said rigid inner layer and said outer layer are independently made from said elastomeric material.

7. The device according claim 1 wherein said device is disposable.

8. A device for sealing an opening of a cervical canal during use of a hysteroscope instrument comprising:
an inflatable housing adapted to be received on an outer portion of a tubular body of said hysteroscope instrument, said inflatable housing surrounding an outer diameter of said outer portion of said tubular body of said hysteroscope instrument; and
means for inflating said inflatable housing, wherein when inflated said inflatable housing provides a seal between said outer portion of said tubular body of said hysteroscope instrument and a wall of said cervical canal.

9. The device according to claim 8 wherein said means for inflating said inflatable housing is locating outside said cervical canal.

10. The device according to claim 8 wherein said device is made from an elastomeric material.

11. The device according to claim 10 wherein said device comprises a plurality of layers.

12. The device according to claim 11 wherein said plurality of layers comprises a rigid inner layer and an outer layer, said rigid inner layer contacts the outer portion of said tubular body of said diagnostic or surgical instrument and said outer layer contacts said cervical canal.

13. The device according to claim 12 wherein said rigid inner layer and said outer layer are independently made from said elastomeric material.

14. The device according to claim 8 wherein said outer portion of said tubular body is a hysteroscope sleeve.

15. The device according to claim 14 wherein said device is permanently attached to said outer portion of a hysteroscope sleeve.

16. The device according to claim 8 wherein said device is disposable.

17. A method of preventing fluid or gas from flowing out of a cervical canal from a uterus during the use of a hysteroscope instrument, the method comprising the steps of:
placing a device with means for inflating said device over a tubular portion of the hysteroscope instrument;
inserting said hysteroscope instrument into the cervical canal of the uterus so that the said device is positioned within the cervical canal and an outer portion of said device is adjacent to walls of the cervical canal; and
inflating said device with said means for inflating said device to form a seal between the outer portion of said device and the walls of the cervical canal for preventing the fluid or gas from the uterus from flowing out of the cervical canal during the use of said hysteroscope instrument.

18. The method according to claim 15, further comprising the step of:
deflating said device and withdrawing the hysteroscope instrument through the cervical canal of the uterus.

19. The method according to claim 16, further comprising the steps of:
disconnecting said means for inflating said device;
removing said device from the hysteroscope instrument; and
disposing of said device.
